(19) United States
(12) Patent Application Publication
(10) Pub. No.: US 2022/0323117 A1
(43) Pub. Date: Oct. 13, 2022

(54) SPINAL FACET IMPLANT AND DELIVERY TOOLS

(71) Applicant: PROVIDENCE MEDICAL TECHNOLOGY, INC., Pleasanton, CA (US)

(72) Inventors: Christopher U. Phan, Dublin, CA (US); Shigeru Tanaka, Half Moon Bay, CA (US)

(21) Appl. No.: 17/620,943

(22) PCT Filed: Jun. 18, 2020
(86) PCT No.: PCT/US2020/038447

§ 371 Date: Dec. 20, 2021

Related U.S. Application Data
(60) Provisional application No. 62/864,814, filed on Jun. 21, 2019.

(51) Int. Cl. A61B 17/70; A61F 2/44; A61F 2/46

(52) U.S. Cl. CPC A61B 17/7064; A61F 2/447; A61F 2/4611; A61F 2002/30774

(57) ABSTRACT

A spinal implant for implantation within a spinal facet joint, the implant comprising: a main body including: opposing top and bottom surfaces; opposing front and rear surfaces; and opposing side surfaces; at least one retaining feature associated with at least one surface of the main body to frictionally engage the implant within the spinal facet joint; and two secure ment apertures extending through the main body to fixedly secure the implant within the spinal facet joint.

### CROSS REFERENCE TO RELATED APPLICATIONS

This application claims the benefit of U.S. Pat. Appl. No. 62/864,814 filed on 21 Jun. 2019 which is hereby incorporated by reference.

### FIELD

The present disclosure relates generally to spinal distraction, and more specifically to devices and methods related to use of a spinal implant to distract a spinal facet joint.

### BACKGROUND

Chronic back problems are one of the most common causes of pain and disability in the United States and other developed countries, and they account for enormous economic costs. According to at least one estimate, spinal fusion procedures, in which two adjacent vertebrae are fused together using plates, screws and other implants, are the most commonly performed surgical procedures in the United States. Spinal fusion is often performed in conjunction with an attempt to increase space between the two adjacent vertebrae being operated on (spinal distraction) and to thus prevent impingement of the spinal cord or nerve roots branching from the spinal cord and passing through openings in the vertebral column (radiculopathy). Unfortunately, most techniques and devices used for performing spinal fusion are relatively invasive and involve a number of risks and difficult recovery and rehabilitation. One of the reasons that spinal fusion surgery is often very invasive is that, due to the position of the spinal cord in back of (posterior to) the central vertebral bodies of spine, many of the procedures require entering the patient through the front of the body (an “anterior approach”) and dissecting through various tissues to gain access to the spine. Fusion procedures are often performed on the cervical spine (neck region), which requires dissecting through the neck, or the lumbar spine (lower back region), which requires dissecting through the abdomen. In either case, cutting through the anterior tissues of the patient to reach the spine is not without risk. Fusion procedures may also involve relatively large plates and screws, which require a relatively large surgical access field and thus more dissection of tissue than would be ideal. Not only are these invasive spinal fusion techniques potentially risky, but they are also expensive and typically require lengthy recovery and rehabilitation times.

Therefore, a need exists for alternative devices and methods for treating spinal instability and spinal stenosis, particularly via fusion of adjacent vertebrae. Such devices and methods may be minimally invasive or less invasive than many of the currently available techniques. For example, it may be advantageous to have devices and methods that use a posterior approach for accessing the spine. At least some of these objectives will be met by the embodiments described below.

### SUMMARY

Embodiments described herein address the challenges described above by providing a system for delivering an implant.

In some aspects, a system for delivering a spinal implant into a spinal facet joint space via a posterior approach is described. In some aspects, the system includes a spinal implant comprising first and second securement apertures configured to receive a fastener to fixedly secure the implant within the spinal facet joint. In some aspects, the system includes a first tool defining two longitudinally extending lumens and comprising a proximal end and a distal end, the spinal implant received at the distal end. In some aspects, the system includes a driver tool received in one of the two longitudinally extending lumens and configured to engage the spinal implant during delivery of the spinal implant to the spinal facet joint space. In some aspects, the system includes a second tool defining a longitudinally extending shaft and the fastener. In some aspects, the second tool is received in one of the two lumens of the first tool to deliver the fastener to one of the securement apertures of the spinal implant.

In some aspects, the system includes a guide tube defining a longitudinally extending lumen and configured to receive at least the first tool to deliver the spinal implant to the spinal facet joint space.

In some aspects, the system includes a third tool that is received in the other of the two lumens of the first tool to deliver a second fastener to the other of the two securement apertures of the spinal implant.

In some aspects, the fastener is a bone screw. In such aspects, the second tool and/or the third tool includes a breakable junction between the longitudinally extending shaft and the bone screw such that the bone screw detaches from the shaft after deployment in one of the securement apertures of the spinal implant.

In some aspects, the implant includes a main body comprising opposing top and bottom surfaces, opposing front and rear surfaces, opposing side surfaces, and having a longitudinal axis extending through the main body. In some aspects, the first securement aperture extends through at least a portion of the rear and top surface. In some aspects, the second securement aperture extends through at least a portion of the rear and bottom surface. In some aspects, the first and second securement apertures extending through the main body at offset angles relative to the longitudinal axis of the main body.

In some aspects, the implant includes at least one engagement feature defined at least partially in the rear surface and at least one of the top or bottom surface of the main body, the engagement feature configured to receive a corresponding engagement feature on the first tool. In some aspects, the at least one engagement feature defined on the main body is a keyway feature and the corresponding engagement feature on the first tool is a corresponding key. In some aspects, the at least one engagement feature is a symmetrical groove, an asymmetrical groove or indentations that is complementary to at least one engagement feature on an implant delivery device. In some aspects, the at least one engagement feature is a protrusion in the rear surface that is complementary to at least one engagement feature on an implant delivery device.

In some aspects, the implant includes at least one retaining feature associated with at least one of the top or bottom surfaces of the main body to frictionally engage the implant within the spinal facet joint.

In some aspects, a spinal implant for implantation within a spinal facet joint is described. In some aspects, the implant includes a main body including opposing top and bottom surfaces; opposing front and rear surfaces; opposing side surfaces; and a longitudinal axis extending through the main body. In some aspects, the spinal implant includes at least one retaining feature associated with at least one of the top or bottom surfaces of the main body to frictionally engage the implant within the spinal facet joint. In some aspects, the spinal implant includes a first securement aperture extending through at least a portion of the top surface. In some aspects, the spinal implant includes a second securement aperture extending through at least a portion of the bottom surface. In some aspects, the first and second securement apertures extending through the main body are at offset angles relative to the longitudinal axis of the main body. In some aspects, the spinal implant includes at least one engagement feature defined at least partially in the rear surface and at least one of the top or bottom surface of the main body, the engagement feature configured to receive an implant delivery device.

In some aspects, the two securement apertures are operable to receive fasteners, preferably a bone screw.

In some aspects, the spinal implant includes at least one window. In some aspects, the spinal implant includes preferably two windows, defined in the top and/or bottom surface of the main body. In some aspects, the spinal implant includes one window defined in each side surface of the main body.

In some aspects, the at least one engagement feature is a keyway. In some aspects, the at least one engagement feature is a symmetrical groove, an asymmetrical groove or indentations that is complementary to features on an implant delivery device.

In some aspects, a system for delivering a spinal implant into a spinal facet joint space via a posterior approach is described. In some aspects, the system includes a spinal implant. In some aspects, the system includes a first tool defining two longitudinally extending lumens and comprising a proximal end and a distal end, the spinal implant received at the distal end. In some aspects, the system includes a driver tool received in one of the two longitudinally extending lumens and is configured to engage the spinal implant during delivery of the spinal implant to the spinal facet joint space. In some aspects, the system includes a second tool defining a longitudinally extending shaft and a fastener. In some aspects, the second tool is received in one of the two lumens of the first tool to deliver the fastener to one of the securement apertures of the spinal implant.

In some aspects, the system includes a third tool is received in the other of the two lumens of the first tool to deliver a second fastener to the other of the two securement apertures of the spinal implant.

In some aspects, the fastener is a bone screw. In such aspects, the second tool and/or the third tool includes a breakable junction between the longitudinally extending shaft and the bone screw such that the bone screw detaches from the shaft after deployment in one of the securement apertures of the spinal implant.

In some aspects, a method of fusing adjacent vertebra defining a spinal facet joint is described. In some aspects, the method includes distracting a spinal facet joint using a first tool with a lumen extending through a main body of the first tool. In some aspects, the method includes inserting a second tool through a first interior channel formed in a third tool, the third tool comprising the first interior channel and a second interior channel. In some aspects, the method includes aligning an engagement feature on a rear face of a spinal implant with a corresponding engagement feature on the third tool. In some aspects, the method includes securing the spinal implant to a distal end of the second tool. In some aspects, the method includes inserting the second tool, third tool, and spinal implant through the lumen of the first tool to deliver the implant to a spinal facet joint. In some aspects, the method includes implanting a spinal implant into the spinal facet joint. In some aspects, the method includes engaging at least one retaining feature positioned on the spinal implant to frictionally engage the spinal implant within the spinal facet joint and removing the second tool. In some aspects, the method includes inserting a fourth tool and securing two securement features extending through the main body of the spinal implant to at least two of the adjacent vertebra to fixedly secure the spinal implant within the spinal facet joint.

In some aspects, the method includes removing the fourth tool from the third tool through the first interior channel. In some aspects, the method includes inserting the fourth tool through the second interior channel of the third. In some aspects, the method includes securing the second securement feature extending through the main body of the spinal implant to a second adjacent vertebra to fixedly secure the spinal implant within the facet joint. In some aspects, the method includes removing the first tool, third tool, and fourth tool from the facet joint and leaving the implant secured to the two adjacent vertebra.

These and other aspects and embodiments will be described in further detail below, with reference to the attached drawing figures.

### BRIEF DESCRIPTION OF THE DRAWINGS

The accompanying drawings, which are incorporated into and constitute a part of the specification, illustrate embodiments of the disclosure and, together with the general description above and the detailed description below, serve to explain the principles of these embodiments.

FIG. 1 is a perspective view of a cage delivery system positioned in a first position in accordance with an embodiment of the present disclosure.

FIG. 2 is a perspective view of the cage delivery system in a second position in accordance with an embodiment of the present disclosure.

FIG. 3 is an enlarged perspective view of a portion of FIG. 2 in accordance with an embodiment of the present disclosure.

FIG. 4 is an exploded view of a cage delivery instrument and an implant in accordance with an embodiment of the present disclosure.

FIG. 5 is an enlarged perspective view of a portion of a cage delivery instrument in accordance with an embodiment of the present disclosure.

FIGS. 6-10 are various views of an implant in accordance with an embodiment of the present disclosure.

FIGS. 11-12 are left rear or distal and right rear or distal perspective views of a cage holder in accordance with an embodiment of the present disclosure.

FIG. 13 is a top view of a cage holder engaged with an implant in accordance with an embodiment of the present disclosure.

FIG. 14 is a perspective cross-sectional view along line 14-14 of the cage holder and implant of FIG. 13 in accordance with an embodiment of the present disclosure.

FIG. 15 is a perspective cross-sectional view along line 15-15 of the cage holder and implant of FIG. 13 in accordance with an embodiment of the present disclosure.

FIG. 16 is a perspective proximal end view of the cage delivery instrument with the release driver shown in a first position in accordance with an embodiment of the present disclosure.

FIG. 17 is an assembled cross-sectional view of the distal end of the cage delivery instrument and implant, with the release driver shown in an alternate position in accordance with an embodiment of the present disclosure.

FIG. 18 is a partially exploded view of the distal end of the cage delivery instrument, release driver, and implant, with the release driver shown in an assembled position in accordance with an embodiment of the present disclosure.

FIG. 19 is an assembled view of the components of FIG. 18 in accordance with an embodiment of the present disclosure.

FIG. 20 is an assembled cross-sectional view of the distal end of the cage delivery instrument and implant, with the bone screw delivery device shown in an first position in accordance with an embodiment of the present disclosure.

FIGS. 21-24 are various views of the implant as positioned in a final trajectory in accordance with an embodiment of the present disclosure.

FIG. 25 illustrates a method of fusing adjacent vertebrae in a spinal facet joint in accordance with one embodiment.

FIGS. 26-29 are various views of an implant in accordance with an embodiment of the present disclosure.

FIG. 30 is a perspective view of a cage holder in accordance with an embodiment of the present disclosure.

FIG. 31 is an enlarged perspective view of a portion of the cage holder of FIG. 30 in accordance with an embodiment of the present disclosure.

FIG. 32 is a perspective cross-sectional view along line 32-32 of the cage holder of FIG. 31 in accordance with an embodiment of the present disclosure.

FIG. 33 is a perspective cross-sectional view along line 33-33 of the cage holder of FIG. 31 in accordance with an embodiment of the present disclosure.

FIG. 34 is a proximal end view of the cage holder of FIG. 30 in accordance with an embodiment of the present disclosure.

### DETAILED DESCRIPTION

Aspects of the present disclosure generally involve devices and methods for treating spinal instability, spinal stenosis and radiculopathy. Spinal stenosis reflects a narrowing of one or more areas of the spine, often in the upper or lower back. This narrowing can put pressure on the spinal cord or on the nerves that branch out from the compressed areas (radiculopathy). Individual vertebrae of the spine are positioned relative to each other, and their separation is maintained by discs separating main vertebral bodies and by capsules positioned within facet joints. The discs and capsules are separated from the bone of their respective joints by cartilage. Spinal stenosis is often indicative of degeneration of a disc, a capsule, or the cartilage in a joint, which leads to a compression of the joints and the narrowing mentioned.

Various embodiments of a device, system and method are described herein for posterior fixation of two adjacent vertebrae of a spine, in an effort to ameliorate spinal instability, spinal stenosis and radiculopathy. Some embodiments involve delivery of the fixation device or implant from a posterior approach using a delivery device.

Lateral mass screws and rods are commonly used in spine surgery as a means for cervical posterior fixation. The system spans multiple levels of the spine to fixate the desired posterior cervical construct. The procedure is considered invasive and undesirable if the patient's morbidity does not require additional posterior procedure(s) such as a laminectomy or foraminotomy.

In addition, there are challenges in placing lateral mass screws in the posterior cervical spine such as ensuring proper trajectory across the lateral mass, preventing bone breach which may cause nerve or tissue damage, properly anchoring the screws in bone to prevent screw backout, and adequate fixation due to poor bone quality.

The devices and methods disclosed herein describe less invasive ways to provide posterior fixation and fusion by placing a cage implant within the intra-facet of the cervical spine and adding additional screws that interface with the cage to fixate the spanning construct of the superior and inferior articular surfaces of the facet joint. Multiple cages and screws can span one or multiple levels of the posterior cervical construct. This can be achieved by placing the cages and screws bilaterally at each cervical facet level that is desired to be fixated.

The cage implant distracts or maintains the intra-facet space thereby reducing or preventing radiculopathy by relieving the impinged lateral nerve root of the treated cervical level. In addition, the cage is wedged in the facet space, immobilizing the motion of the treated construct to provide fixation needed for cervical fusion. In certain embodiments, the two screws that interface with the cage also anchor into the superior and inferior articular surfaces to provide additional fixation to further reduce motion of the treated level to promote fusion.

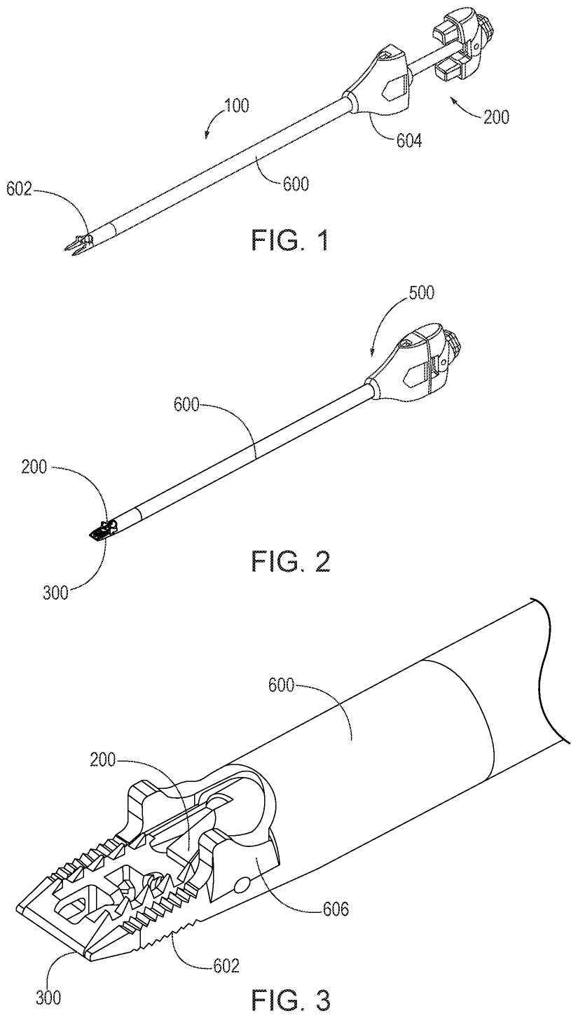

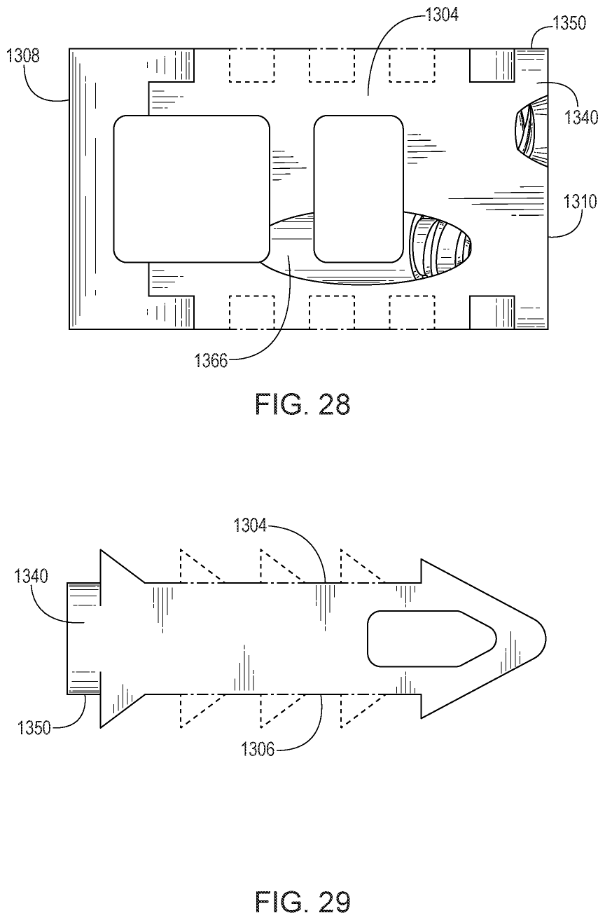

Turning now to the figures, FIGS. 1-24 and 26-34 illustrate various embodiments of a spinal cage delivery system operable to fixedly engage two adjacent vertebrae of a spinal facet joint to fuse two adjacent vertebrae together (e.g., vertebrae of the human cervical spine, such as the C5 and C6 vertebrae.) FIG. 1 is a perspective view of a cage delivery system 100 positioned in a first position. FIG. 2 is a perspective view of the cage delivery system 100 in a second position. FIG. 3 is an enlarged perspective view of a portion of FIG. 2.

The cage delivery system 100 may be used with a cannulated instrument, such as a guide tube, to provide a minimally disruptive way of accessing the surgical site and deploying the cage and screws through the instrument's cannulated channel.

In some examples, the cage delivery system 100 includes a cage delivery instrument 500, a guide tube 600, and an implant or cage 300. The cage delivery instrument 500 may include a cage holder 200, and a release driver 400, and the system 100 or instrument 500 may further include a bone screw delivery device 700.

In some examples, a proximal end 604 of the guide tube 600 may be formed as a handle. The distal end 602 of the guide tube 600 may be formed as forks, arms or protrusions 602 with serrated surfaces. Adjacent the forks 602 may be a raised or stop feature 606, that when contacting a vertebrae, may help to prevent from the over-advancement of the end 602 of the guide tube 600 into the target location.

The guide tube 600 may be formed as a hollow tube or longitudinally extending lumen through which various tools, such as a cage holder 200 and implant 300 may be inserted through to access a target location.

In use, to prepare for deployment, the release driver 400 may be inserted through the cage holder 200. A distal end of the release driver may extend past the distal end of the cage holder 200, and the implant 300 may be detachably coupled to the distal end of the release driver. The guide tube 600 may then be deployed to a target location, for example a facet joint, such as a cervical facet joint. Once deployed, the implant 300, positionably coupled to the cage holder 200 via the release driver 400, is inserted through the hollow tube of the guide tube 600, until the implant 300 reaches the target location. Once the implant reaches the target location, the release driver 400 may be disengaged or decoupled from the implant 300, and the release driver may then be removed from within the cage holder. A bone screw delivery device 700, with a distal region 704 including another implant or fastener, such as a bone screw 708, may then be inserted through the cage holder to be coupled to the implant 300. Using the bone screw delivery device, the bone screw 708 may engage with threaded holes of the implant and also engage with adjacent surfaces of the facet joint. The bone screw 708 may then be separated from the shaft 706 of the bone screw delivery device 700 upon the distal region 704 experiencing a predetermined load, as described in more detail below. The cage holder 200, guide tube 600 and remaining portion of the bone screw delivery device 700 (i.e. the shaft portion 706) may be removed from the target location, leaving the implant 300 and the bone screw 708 in place.

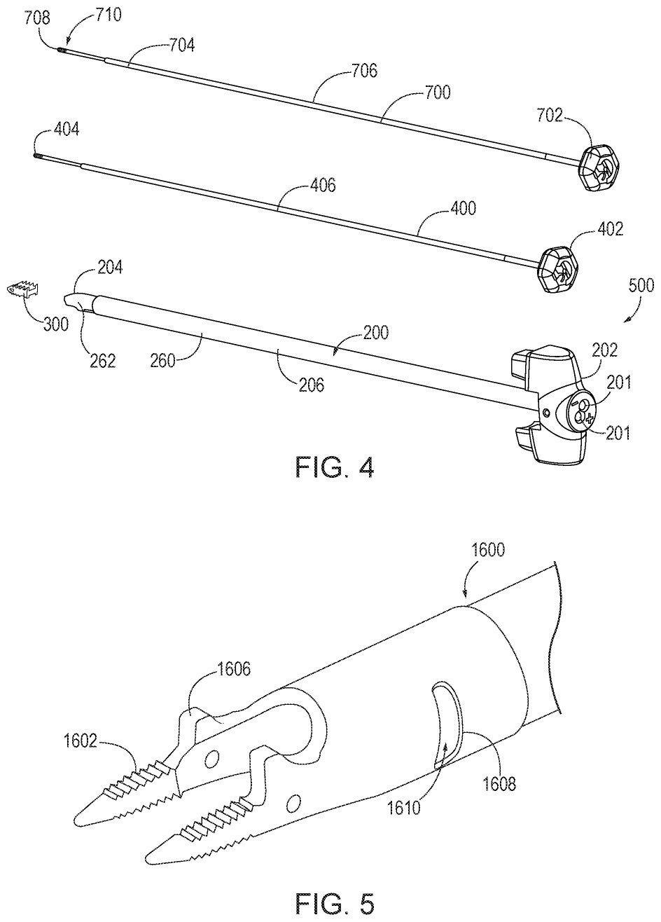

FIG. 4 is an exploded view of a cage delivery instrument 500 and an implant 300 in accordance with an embodiment of the present disclosure. The cage delivery instrument 500 may include a release driver 400, and a cage holder 200, and in some aspects, may also include or be used with a bone screw delivery device 700.

The release driver 400 may include a proximal end formed by a driver handle 402, and a threaded distal end 404, and a shaft coupling the driver handle 402 and threaded distal end 404. The bone screw delivery device 700 may include a proximal end including a handle 702, and a distal region 704 including a bone screw 708, and a shaft coupling the handle 702 to the distal region 704. A breakable junction 710 is defined between the shaft and the bone screw.

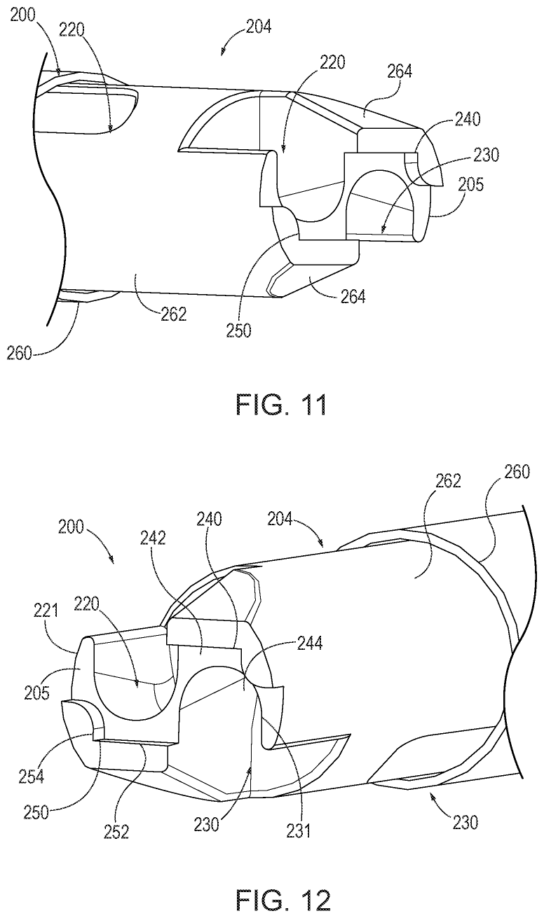

The cage holder 200 may include a proximal end or portion formed by a delivery instrument handle 202 and a distal end or portion 204 opposite the handle 202. The handle 202 may be received by and used to couple with a handle of the guide tube 600. The shaft 206 may couple the delivery instrument handle 202 and distal end 204. In some examples, as explained with reference to FIGS. 11-17, the cage holder 200 includes two generally parallel but offset lumens or internal channels 220, 230 (see FIG. 11) that extend along the interior of shaft 206 between the handle and the distal end 204. In some examples, the channels 220, 230 may form a double barrel configuration. The handle 202 may include two openings or holes 201 connected to and providing access to the lumens 220, 230. The release driver 400 can be placed down either handle hole 201 on the cage holder 200. The driver is guided by the lumens or internal channels on the holder shaft and exits at the holder's distal tip. When assembled, the implant 300 may be positioned adjacent the distal end 204 of the cage holder 200, and the release driver 400 may be inserted through the cage holder 200, with the threaded distal end 404 of the release driver 400 coupled to implant 300. The release driver may then be decoupled from the implant 300, and the bone screw delivery device 700 may be inserted through the cage holder 200, with the bone screw 708 then coupled to the implant.

FIG. 5 is an enlarged perspective view of a cage delivery instrument in accordance with an embodiment of the present disclosure. The guide tube 1600 of FIG. 5 may include features similar to that of guide tube 600. Guide tube 1600 may include a distal end 1602 that may be formed as forks, arms or protrusions with serrated surfaces. Adjacent the forks 1602 may be a raised or stop feature 1606, that when contacting a vertebrae, may help to prevent from the over-advancement of the end 1602 of the guide tube 1600 into the target location.

The guide tube 1600 may be formed as a hollow tube or longitudinally extending lumen through which various tools, such as a cage holder 200, 1200 and implant 300, 1300 may be inserted through to access a target location. In some embodiments, the guide tube 1600 may also include an aperture or cutout 1610. The cutout 1610 is located on the tip portion of the guide tube that is welded to the main shaft. The cutout is used for additional welding in addition to a circumferential seam weld.

In some embodiments, the aperture or cutout 1610 of the guide tube 1600 may extend through a sidewall 1608 of the guide tube adjacent the distal end 1602. In use in this embodiment, the cutout 1610 may allow for the visual confirmation of the tool's position as it is extended through the lumen of the guide tube 1600. For example, a practitioner could visually see the position of a cage holder 200 and implant 300 within the guide tube 1600 as the tools are inserted through the lumen of the guide tube to access a target location.

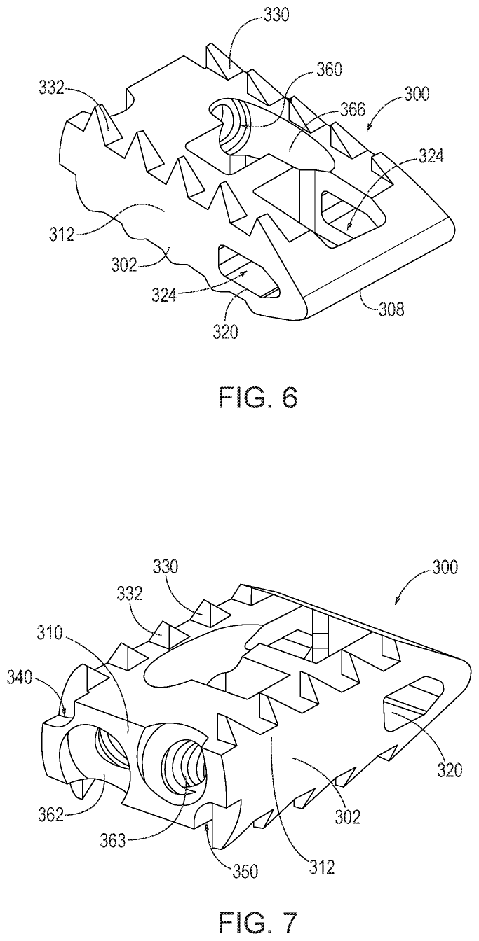

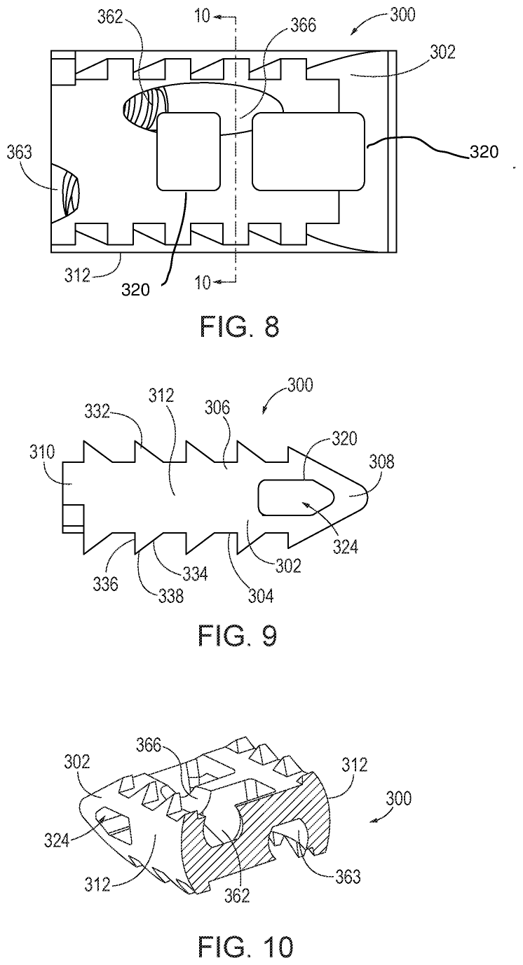

FIGS. 6-10 are various views of an implant 300 in accordance with an embodiment of the present disclosure. FIG. 6 is a front perspective view of the implant 300. FIG. 7 is a rear perspective view of the implant 300. FIG. 8 is a top plan view of the implant 300. FIG. 9 is a side elevation view of the implant 300. FIG. 10 is a cross-sectional view along line 10-10 of the implant 300 of FIG. 8.

The implant 300 may include a main body 302 defined by opposing top and bottom surfaces 304, 306, opposing front and rear surfaces 308, 310, and opposing side surfaces 312. In some embodiments, some of the surfaces (e.g., the opposing top and bottom surfaces 304, 306) may be generally planar. The implant 300 may be generally cuboid in shape, though other shapes are contemplated that permit the implant 300 to be inserted within a spinal facet joint and maintain a certain distance between two adjacent vertebrae. The front surface 308 may be rounded in shape and have a tapered or bull-nosed shaped profile.

In some examples, as shown in FIG. 10, the side surfaces 312 may be convex in shape, or have a rounded or arcuate profile. In some examples, this may allow the width of the implant between the sides 312 to be maximized while still allowing the implant to be deployed through the various instrumentation described. As described in more detail below, the spinal implant 300, which may be formed of a bone or bone substitute material or a biocompatible metal (e.g., titanium), ceramic, polymer, or some combination thereof, may be sized and shaped to fit snugly (e.g., through friction fit) into or otherwise engage or abut adjacent vertebrae of the spinal facet joint.

To reduce weight and offer cross-sectional areas for new bone growth and fusion, for instance, the implant 300 may include one or more windows 320 defined in at least one surface of the main body 302. For example without limitation, the implant 300 of FIGS. 6-10 includes a two windows 320 defined in each of the top and bottom surfaces and a single window in each opposing side surfaces 312 of the main body 302, though any number of windows 320 is contemplated. The windows 320 may be any size, shape, and orientation. As shown, each of the windows 320 is adapted to place at least a portion of a hollow interior of the implant 300 in communication with the surrounding environment.

In such embodiments, a hollow interior of the implant 300 may include one or more chambers, such as a distal chamber 324 positioned near the front of the implant 300. To permanently fuse adjacent vertebrae together, the chambers 324 may by packed (via the windows 320, for instance) with a bone or bone substitute material to cause bone ingrowth into the hollow interior of the implant 300.

With continued reference to FIGS. 6-10, the implant 300 may include at least one retaining feature 330 associated with at least one surface of the main body 302 to frictionally engage the implant 300 within a spinal facet joint.

For instance, the at least one retaining feature of the implant 300 may include a plurality of protrusions 332 extending away from at least one of the opposing top and bottom surfaces 304, 306 of the main body 302 (e.g., from both the top and bottom surfaces 304, 306). As described herein, the protrusions 332, which may be referred to as teeth, may be operable to permit the implant 300 to be inserted into a spinal facet joint but may also limit its removal therefrom. For example, the protrusions 332 may be directionally sized and shaped such that a force required to remove the implant 300 from the spinal facet joint is substantially greater than a force required to insert the implant 300 within the facet joint. In this manner, the implant 300 may be inserted into proper position within the facet joint as desired. Once inserted, the protrusions 332 may limit movement of the implant 300 within the facet joint in at least the removal direction. In some embodiments, the protrusions 332 may be operable to limit lateral movement of the implant 300 within the facet joint.

As shown in FIG. 9, each of the protrusions 332 may include a leading face 334, a trailing face 336, and a tip 338 formed at an intersection between the leading and trailing faces 334, 336. In some embodiments, the protrusions 332 may extend from adjacent (e.g., at or near) an edge 340 defined between the opposing top and bottom surfaces 304, 306 and the opposing side surfaces 312. In such embodiments, each of the top and bottom surfaces 304, 306 may include two rows of protrusions 332 extending between the front and rear surfaces 308, 310 and adjacent (e.g., along) opposing edges 340 of the respective surface, the window 320 being positioned between the rows of protrusions 332 on each side. As shown in FIG. 9, each row of protrusions 332 may include a saw tooth profile, though other profile shapes are contemplated including triangle, square, and sinusoidal, among others.

In addition to the description above, the protrusions 332 may be variously sized and shaped in other ways. For instance, the height of the protrusions 332 (as defined by the tips 338) may be uniform or may vary along the length of the implant 300 between the front and rear surfaces 308, 310 of the main body 302. For instance, the protrusions 332 positioned nearer the front surface 308 of the main body 302 may have a smaller height than the protrusions 332 positioned away from the front surface 308 (see FIG. 9) or vice-versa. Similarly, the distance between the protrusions 332 may be uniform or may vary along the length of the implant 300. For instance, the distance between the protrusions 332 positioned nearer the front surface 308 may be less than the distance between the protrusions 332 positioned nearer the rear surface 310, or vice-versa. In some embodiments, and in order to accommodate the mating geometry of the rear face, the protrusions 332 do not extend the entire length of the implant or at least such protrusions 332 are not positioned on the at least one engagement feature (protrusion extending from the rear or proximal face) but rather are positioned near or adjacent the at least one engagement feature, as described below. In some embodiments, the trailing face 336 of the protrusion(s) closest to the rear face are not aligned with or coplanar with the rear surface of the implant. Instead, the protrusion (and its trailing face) is a distance away from or spaced apart from the rear face.

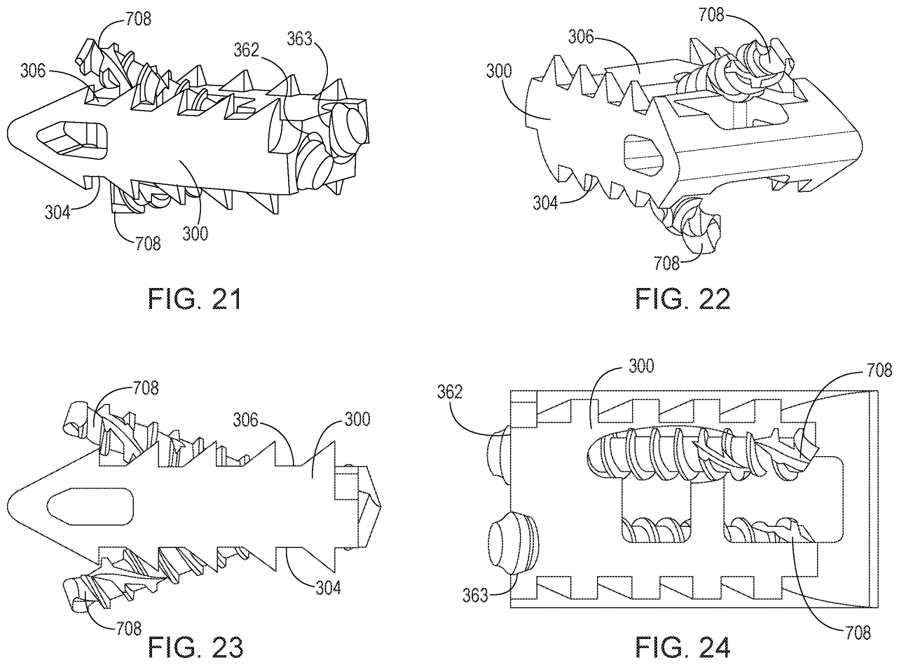

With continued reference to FIGS. 6-10, the implant 300 may include at least one securement feature or aperture 360 associated with at least one surface of the main body 302 to fixedly secure the implant 300 within the spinal facet joint. For instance, at least one securement aperture may be defined in the main body 302 (e.g., in at least the rear surface 310 of the main body 302), the securement aperture operable to receive a fastener therein, such as the threaded distal end 404 of the release driver 400 and/or the bone screw 708 of the bone screw delivery device 700. As shown in FIG. 7, the securement aperture 362 or securement aperture 363 may be angled such that the bone screw 708 would extend through the rear surface 310 and one of the top and bottom surfaces 304, 306 of the main body 302 to engage an adjacent vertebra. In some examples, the implant 300 includes two securement apertures 362, 363 with a first securement aperture 362 extending through the rear surface 310 and the top surface 304, while the second securement aperture 363 extends through the rear surface 310 and through the bottom surface 306. FIGS. 21-25 provide additional views of the implant with fasteners positioned within the securement apertures.

With continued reference to FIGS. 7 and 10, the rear face includes at least one engagement feature defined at least partially in the rear surface and at least one of the top or bottom surface of the main body, the engagement feature configured to receive a corresponding engagement feature on the first tool. More specifically, the rear face or surface 310 of the implant 300 may have mating geometry that engages the tip 205 of the distal end 204 of the cage holder 200 and may also include mating geometry to engage the bone screw 708 of the bone screw delivery device 700. This mating geometry may help prevent rotation and translational movement when the cage or implant 300 is attached or coupled to the cage holder 200. The engagement feature of the implant may include a raised or recessed area complimentary to an engagement feature on the cage holder. For example, the engagement feature on the implant may include a symmetrical or asymmetrical protrusion on or near the rear surface of the implant that is configured to positively engage or be positioned with a symmetrical or asymmetrical groove, recess or counter bore feature on the cage holder. In an example, the engagement feature or protrusion of the implant may seat at least partially within the recess of the cage holder. The protrusion may also have rounded corners and the securement apertures may be at least partially defined in the protrusion.

The mating geometry of the rear face 310 of the implant 300 may include an additional engagement feature, such as keyways 340, 350, as shown in FIG. 7. In some examples, the keyway features 340,350 are asymmetrical grooves or indentations that are complementary to features on the distal end 204 of the cage holder 200. In some examples, the implant 300 only includes a single keyway 340 or keyway 350. As shown in FIG. 7, the keyway 350 may be located adjacent the securement aperture 363, and the keyway 340 may be located adjacent the securement aperture 362.

To aid in securing the implant 300 within the facet joint, the securement aperture 362 may be angled so the bone screw inserted therein extends upwardly to engage an upper or superior vertebra and a second bone screw inserted therein extends downwardly to engage a lower or inferior vertebrae. In some applications, only one of the angled securement apertures 362 may be engaged. In use, a bone screw may then be positioned within each aperture 362 to engage adjacent vertebrae. The angled securement apertures may help dictate the trajectory of the screw, and may also help provide an anchor utility to couple or anchor the screws to the implant or cage 300. The angled securement apertures may also be used to attach or couple the cage or implant 300 to the cage delivery instrument 500. FIGS. 21-25 provide additional views of the implant with fasteners positioned within the securement apertures. In some embodiments, the securement aperture 362, 364 may be a straight, non-angled securement aperture. In other embodiments, the securement aperture 362, 364 may be a longitudinal, non-angled securement aperture.

The implant 300 may be inserted within a patient's facet joint irrespective of the relative positions of the top and bottom surfaces 304, 306. As shown in FIGS. 6 and 8, the main body 302 may include a notch or open channel 366 on both the top and bottom surfaces 304, 306. The channel 366 may extend through an interior wall of the implant 300. The channel 366 may help to at least accommodate the bone screw to be inserted within the implant 300. The channel 366 may help support the screw trajectory when being driven through the cage or implant 300 into bone. The bone screw described herein may be made of any suitable material, including biocompatible metals, ceramics, and/or polymers.

FIGS. 11-17 are various views of the cage holder 200. FIGS. 11-12 are left rear and right rear perspective views of the distal end 204 of cage holder 200 in accordance with an embodiment of the present disclosure. The distal tip 205 of the cage holder 200 may have mating geometry that engages the back of the cage or implant 300 to help prevent rotation and translational movement when the cage or implant 300 is attached. The distal end 204 may include certain engagement features that extend away from the tip 205 of the distal end 204.

In some examples, the engagement features may include key or male feature 240, 250. In some examples, the keyed features 240, 250 are similar or symmetrical with each other. In some examples, the keyed features 240, 250 may differ from each other in at least one feature or aspect.

In an example, the key 240 includes flat portion 242 and rounded portion 244. The combination of the flat portion 242 and rounded portion 244 may form an L-like shape. This may create a positive engagement with female or keyway features 340 adjacent or formed on the rear face 310 on the implant 300. In some examples, the key 250 includes a flat portion 252 and rounded portion 254. The combination of the flat portion 252 and rounded portion 254 may also create an L-like shape to help create a positive engagement with female or keyway features 350 adjacent to or formed on rear face 310 on the implant 300.

In some examples, the key 240, 250 may each extend between about 45 and about 90 degrees radially about an outer circumferences of the distal end 204. In some examples, the radial length or distance of the key 240, 250 is decreased as a chamfer 264 may also extend about the outer circumference, in between the outlets of the first channel 220 and second channel 230.

With continued reference to FIGS. 11 and 12, the cage holder 200 may also include channels 220, 230 that extend down the length of the shaft 206. The outlet 221, 231 of each channel 220, 230 is positioned adjacent the tip 205 of the distal end 204 of the cage holder 200. At the respective outlet, each channel 220, 230 may have a generally u-shaped profile, as the outermost edge of each channel at the outlet is open or unenclosed. Other profile shapes may be used, such as oval, rectangular, or oblong. In addition, the tip 205 may include a chamfer 264 adjacent the outlet 221, 231 of the channels.

As shown in FIGS. 4 and 11-15, the cage holder 200 may include an outer sheath 260 positioned about a main body 262. The outer sheath 260 may be shorter than the overall length of the cage holder 200. In some examples, the outer sheath 260 does not extend past the distal end 204. The outer sheath 260 may be coupled to the main body 262 at the delivery instrument handle 202. The main body 262 may include a first channel 220 and a second channel 230.

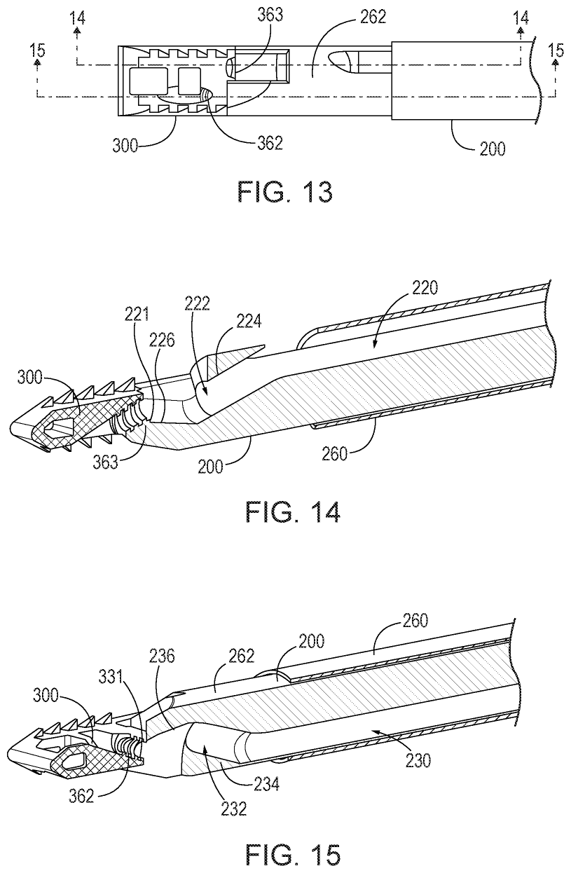

FIGS. 13-15 show additional details regarding the channels 220, 230. FIG. 13 is a top view of a cage holder 200 assembled to an implant 300. FIG. 14 is a perspective cross-sectional view along line 14-14 of the cage holder 200 and implant 300 of FIG. 13. FIG. 15 is a perspective cross-sectional view along line 15-15 of the cage holder 200 and implant 300 of FIG. 13. As shown in FIG. 14, the channel 220 is positioned within a main body of the cage holder until it exits the main outer sheath 260 near the distal end 204. The channel then forms a ramp 222, formed by a first portion 224 and a second portion 226. The first portion 224 of the ramp 222 angles the channel downward, towards a centerline of the cage holder 200. Then, the second ramp 226 decreases or flattens out the alignment of the channel 220 so that the outlet 221 of the channel 220 generally aligns with the aperture 363 of the implant 300. The ramps may cause the distal threaded end 404 of the release driver to exit the distal end of the cage holder 200 at a second trajectory.

As shown in FIG. 15, the channel 230 extends towards the distal end 204 until it exits the main outer sheath 260. The channel 230 then forms a ramp 232, formed by a first portion 234 and a second portion 236. The first portion 234 of the ramp 232 angles the channel 230 upward, towards a centerline of the cage holder 200. Then, the second ramp 236 decreases or flattens out the alignment of the channel 230 so that the outlet 231 of the channel 230 generally aligns with the aperture 362 of the implant 300.

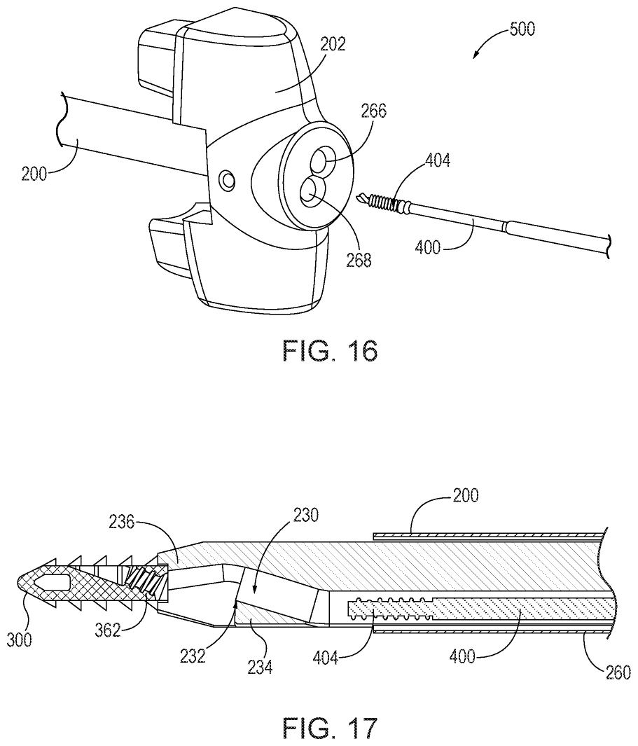

FIG. 16 is a perspective end view of the cage delivery instrument 500 with the release driver 400 positioned in a first position. As shown in FIG. 16, the delivery instrument handle 202 of the cage holder 200 includes apertures 266, 268.

Aperture 266 is the inlet to the channel 220. Aperture 268 is the inlet to the channel 230. In use, the release driver 400 is removed from the cage system 100, exposing the handle holes 266, 268. The release driver 400 and the bone screw delivery device 700, may each be placed down either handle hole 266, 268 and deployed into the implant or cage one at a time or in series. For example, the threaded distal end 404 of the release driver 400 may be inserted through either aperture 266 or aperture 268 to then couple the threaded distal end 404 with the implant. The cage release driver 400 can be first placed down either handle hole 266, 268 in the delivery instrument handle 202. The release driver 400 is guided by the respective internal channel 220, 230 on the holder shaft 206 and exits at the cage holder's distal end 204.

FIG. 17 is an assembled cross-sectional view of the cage delivery instrument 500 and implant 300, with the release driver 400 positioned in an alternate position. As shown in FIG. 17, the ramp 232 near the distal tip of the cage holder 200 angles at first portion 234 and second portion 236 to guide the threaded distal end 404 to the angled trajectory of the thread feature 362 on the cage 300. In other examples, when the release driver 400 is positioned within the channel 220, the ramp 222 near the distal tip of the cage holder angles at a first portion 224 and a second portion 226 to guide the threaded distal end to align with the angled trajectory of the thread feature 363 on the cage 300.

The delivery system 100 may be used to release the implant 300 from the release driver 400 and cage holder 200. Once the implant is positioned, the handle of the release driver may be rotated to disengage the implant from the release driver. The release driver may then be removed and separated from the cage holder 200. In one implementation, the release driver includes engagement features at a distal tip 404 of the shaft. The engagement features may be any feature adapted to engage the securement feature 360 or a feature of the rear surface 310 of the implant 300. For example, the engagement member on the end of the distal tip may be threaded members.

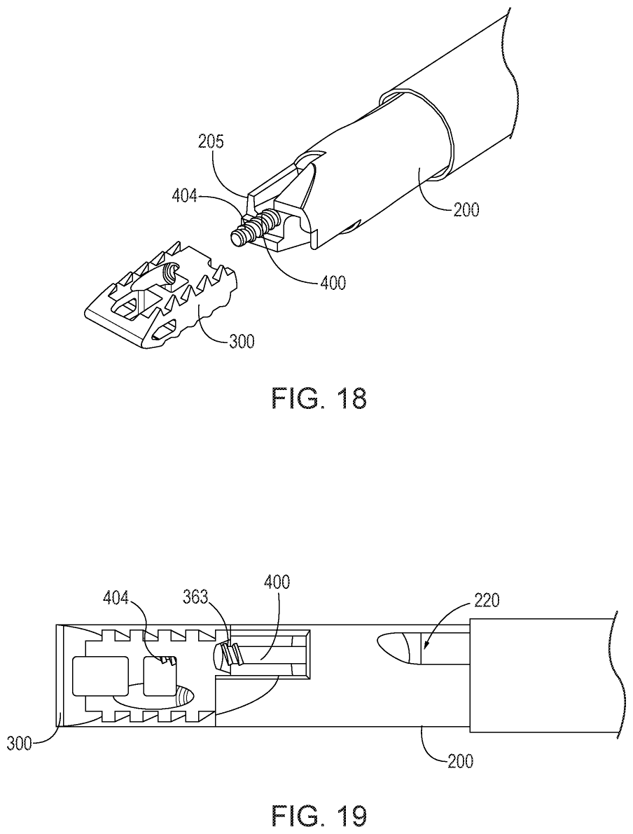

FIG. 18 is a partially exploded view of the cage delivery instrument 500 and implant 300, with the release driver 400 positioned in an assembled position. As shown in FIG. 18, the release driver 400 is inserted through the corresponding handle holes in delivery instrument handle 202 so that the threaded distal end 404 of the release driver 400 extends past the tip 205 of the distal end 204 of the cage holder 200.

FIG. 19 is an assembled view of the components of FIG. 18 in accordance with an embodiment of the present disclosure. In one implementation, the distal threaded portion 404 of the release driver 400 engages with one of the threaded holes 362, 363 on the cage or implant 300 to secure the cage to the cage holder 200 of the cage delivery instrument 500 to allow the implant to be delivered to the target location. FIG. 19 shows the threaded distal end 404 of the release driver 400 engaged with the implant 300. In the embodiment of FIG. 19, the release driver 400 is positioned through the lumen 220 and the screw extends through threaded aperture 363 of the 300. Once the implant is delivered, the release driver 400 is disengaged from the implant, and the release driver is withdrawn through the cage holder. The implant remains positioned in the target location, with the cage holder engagement features engaging the implant. The bone screw delivery device 700 may then be inserted through the cage driver to engage the implant and further secure the implant to the target location.

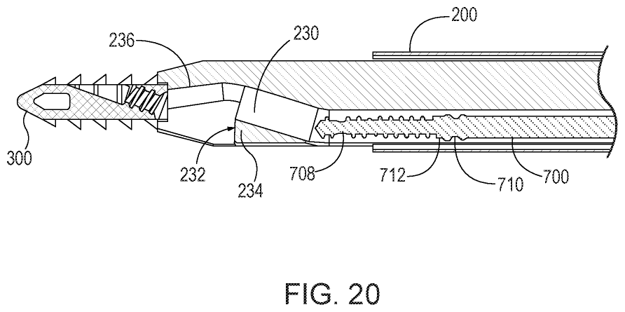

FIG. 20 is an assembled cross-sectional view of the cage delivery instrument 500 and implant 300, with the bone screw delivery device 700 positioned in an first position. Similar to the operation of the release driver being inserted through the cage delivery instrument described in FIG. 17, the ramp 232 near the distal tip of the cage holder 200 angles at first portion 234 and second portion 236 to guide the bone screw 708 of the distal region 704 to the angled trajectory of the thread feature 362 on the cage 300. In other examples, when the distal portion 704 is positioned within the channel 220, the ramp 222 near the distal tip of the cage holder angles at a first portion 224 and a second portion 226 to guide the bone screw 708 to align with the angled trajectory of the thread feature 363 on the cage 300.

The delivery system 100 may include an engagement feature to secure the implant 300 to adjacent vertebrae. In one implementation, the bone screw delivery device 700 includes a flexible shaft or rod 706 and includes a bone screw 708 at a tip of the distal region 704 of the flexible shaft 706. The engagement features may be any feature adapted to engage the securement feature 360 or a feature of the rear surface 310 of the implant 300. For example, the engagement member on the end of the distal tip may be threaded members of the bone screw 708.

The lumens or channels 220, 230 in the cage holder 200 help to guide the bone screw(s) 708 to the rear surface of the implant 300 and the corresponding threaded securement aperture. In use, the bone screw delivery device 700 is rotated or advanced, such as via the handle, and the bone screw 708 engages with the threaded apertures of the implant and into the adjacent vertebra. Upon the bone screw 708 becoming sufficiently secured to the vertebral bone, a breakable junction 710 defined between the shaft 706 and the bone screw 708 experiences a predetermined load to cause the bone screw 708 to detach from the shaft 706 of the bone screw delivery device 700. In some examples, the bone screw 708 is driven into the implant 300 and bone until the head 712 of the screw hard stops against the back surface 310 of the implant. The screw 708 is then snapped or broken off at the designated break-off point or breakable junction 710 along its shaft to complete the deployment.

FIGS. 21-24 are various views of the implant 300 as positioned after the bone screws are in a final trajectory, after the bone screws 708 are driven into and engaged the threaded apertures 362, 363 on the implant 300 as would be the case when positioned in a facet joint. FIG. 21 is a rear perspective view of the implant 300 and two bone screws 708 extending through the angled apertures 362, 363 and past the upper and lower surfaces 304, 306. FIG. 22 is a front perspective view of the implant 300 and two bone screws 708. FIG. 23 is a side elevation view of the implant 330 and bone screws 708. FIG. 24 is a top view of the implant 300 and bones screws 708. In some examples, the implant 300, when positioned in the final trajectory, such as in a facet joint, between two vertebrae, may include no bone screws, one bone screw, or two bone screws. The exit portion of each screw anchors into either the superior or inferior articular process of the intra-facet joint. Screw trajectory can vary based on the desired angle trajectory of the cage threads. In some examples, the screws lock into the cage with mating threads to help prevent screw back-out. In some examples, the facet joint is a cervical facet joint.

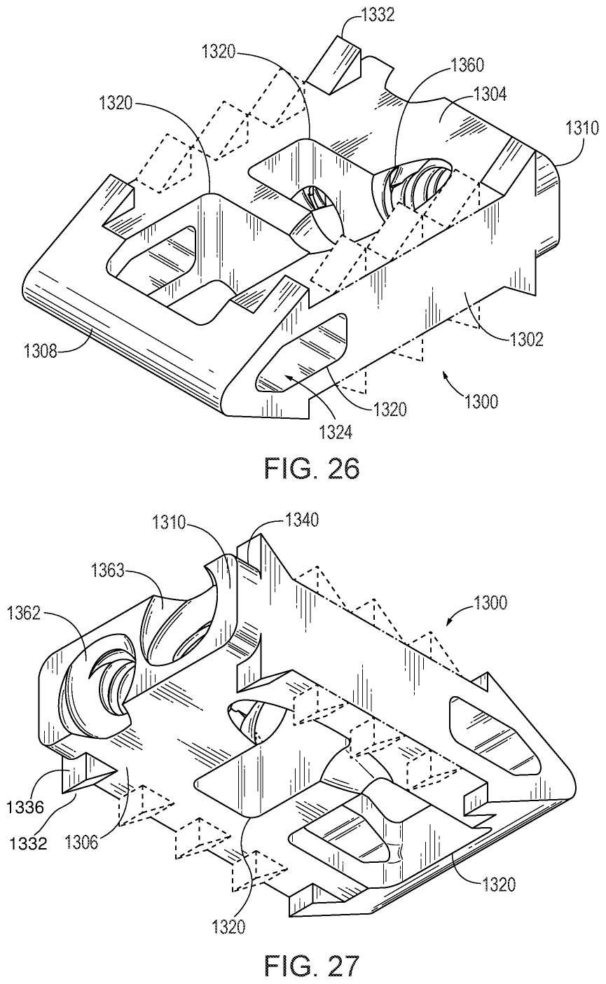

FIGS. 26-29 are various views of an implant in accordance with an embodiment of the present disclosure. The implant 1300 may have features similar to that of implant 300, with a main body 1302, generally planar top and bottom surfaces 1304 and 1306, opposing front and rear surfaces 1308, 1310, and generally planar opposing side surfaces 1312 with a generally cuboid shape. The front surface 1308 may be rounded in shape and have a tapered or bull-nosed shaped profile. A plurality of protrusions 1332 may extend away from at least one of the opposing top and bottom surfaces 1304, 1306 of the main body 1302. Similar to implant 300, each protrusion includes a leading face, a trailing face and a tip defined at the intersection between the faces. For example without limitation, the implant 1300 includes two windows 1320 defined in each of the top and bottom surfaces 1304, 1306 and a single window in each opposing side surfaces 1312 of the main body 1302, though any number of windows 1320 is contemplated.

The rear surface 1310 of the implant 1300 may include an engagement feature configured to receive a corresponding engagement feature on a tool. In some examples, the engagement feature includes mating geometry that engages a tip of a distal end of a cage holder. This mating geometry may help prevent rotation and translational movement when the cage or implant 1300 is attached or coupled to the cage holder 1200. In an example, the engagement feature includes a rectangular protrusion 1340 with rounded corners 1350. The rectangular protrusion 1340 and rounded corners 1350 may align with mating geometry of the cage holder 1200, so that the engagement feature of the implant seats at least partially within a groove, recess or counter-bore feature on the distal end of the cage holder.

The protrusion 1340 with rounded corners may include the securement apertures that may be at least partially defined in the protrusion. In some embodiments, and in order to accommodate the mating geometry of the rear face, the protrusions 1332 do not extend the entire length of the implant or at least such protrusions 1332 are not positioned on the engagement feature (protrusion extending from the rear or proximal face) but rather are positioned near or adjacent the engagement feature. In some embodiments, the trailing face 1336 of the protrusion(s) 1332 closest to the rear face is/are not aligned with or coplanar with the rear surface of the implant. Instead, the protrusion (and its trailing face) is a distance away from or spaced apart from the rear face.

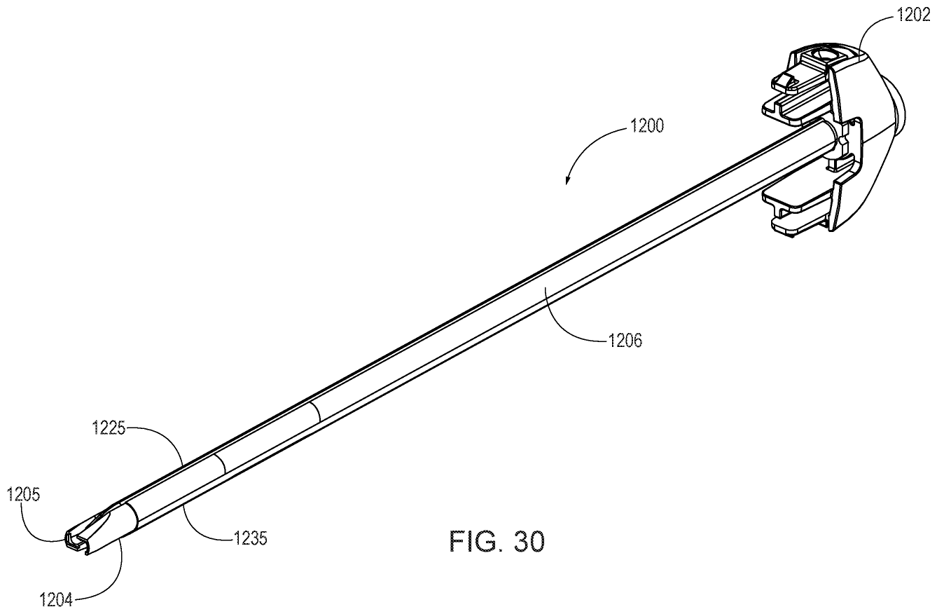

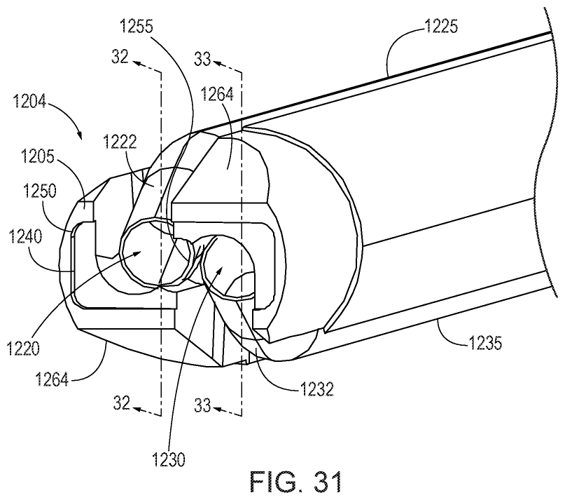

FIGS. 30-34 are various views of a cage holder 1200 in accordance with an embodiment of the present disclosure. FIG. 30 is a perspective view of a cage holder 1200. FIG. 31 is an enlarged perspective view of a portion of the cage holder 1200 of FIG. 30. The cage holder 1200 may include similar features to the cage holder 1200 shown in FIGS. 11-20.

As shown in FIG. 31, the distal tip 1205 of the cage holder 1200 may have mating geometry that engages the rear of the cage or implant 300 to help prevent rotation and translational movement when the cage or implant 300 is attached or coupled to the cage holder. The engagement feature of the cage holder may include a raised or recessed area complimentary to an engagement feature on the implant.

The engagement feature of the cage holder may include a rectangular shaped recess, groove or counter bore 1240, with rounded corners 1250. The recess 1240 may be configured to positively position or engage with the protrusion 1340 of the implant 1300. In use, the protrusion 1340 of the implant may seat at least partially within the recess 1240 of the cage holder.

The cage holder 1200 may also include two generally parallel but offset lumens or channels 1220, 1230 (see FIG. 31) that extend longitudinally along the shaft 1206 between the handle 1202 and the distal end 1204, the channels 1220, 1230 forming a double barrel configuration. In some examples, a hollow tube 1225, 1235 is positioned adjacent or at least partially within the channel 1220, 1230. In some examples, the distal end of the hollow tube has a ramp or slope 1222, 1232 that is angled inward towards a central axis of the shaft 1206. In use, the threaded distal end 404 of the release driver 400 may engage or contact the slope 1222, 1232. This contact may cause the trajectory of the threaded distal end 404 of the release driver 400 to be changed as it is inserted through the cage holder 1200 so that the distal end 404 aligns with the securement apertures of an implant 300, 1300. Similarly, as the distal end 704 of the bone screw delivery device may contact the slope as it is inserted through the channel 1220, 1230. The contact may change the trajectory of the distal end 704 so that it aligns with the securement apertures of an implant 300, 1300.

In some examples, the distal end 1204 of the cage holder may include a chamfer 1264 that extends from the tip 1205 towards the proximal end of the cage holder. The tip 1205 of the cage holder may include an additional recess 1255 positioned between the distal ends of the two tubes 1225, 1235 or channels 1220, 1230. In some examples, the implant may include an additional engagement feature that is configured to engage with recess 1255 when the implant is coupled to the cage holder 1200.

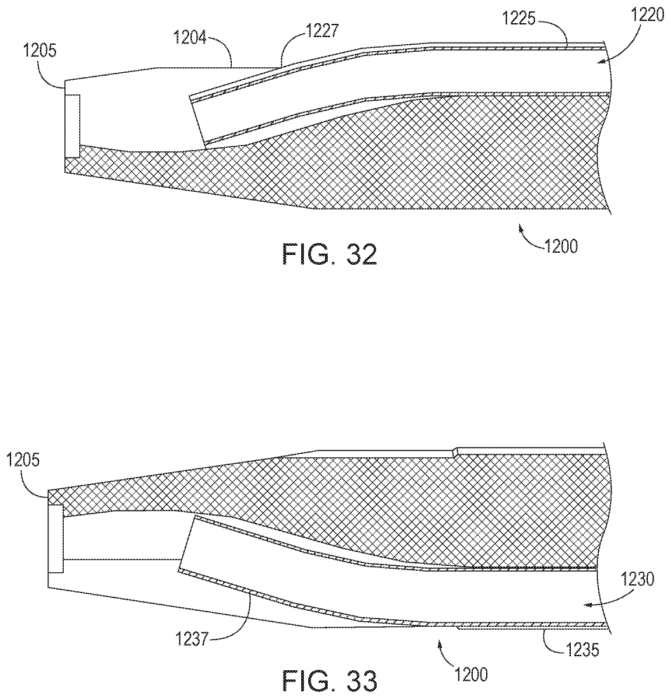

FIG. 32 is a perspective cross-sectional view along line 32-32 of the cage holder of FIG. 31 in accordance with an embodiment of the present disclosure. As shown in FIG. 32, at the distal end 1204 of the cage holder 1200, the tube 1225 includes a ramp or slope 1222 towards a central axis of the cage holder.

FIG. 33 is a perspective cross-sectional view along line 33-33 of the cage holder of FIG. 31 in accordance with an embodiment of the present disclosure. As shown in FIG. 33, at the distal end 1204 of the cage holder 1200, the tube 1235 includes a ramp or slope 1232 towards a central axis of the cage holder.

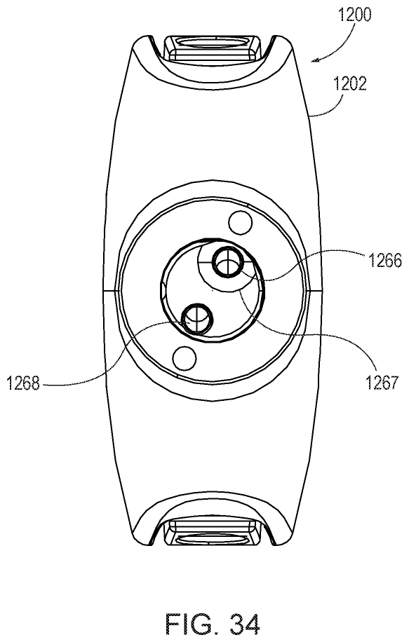

FIG. 34 is a proximal end view of the cage holder of FIG. 30. The handle 1202 of the cage holder 1200 may include two handle holes or apertures 1266, 1268 connected to and providing access to the lumens 1220, 1230 and/or tubes 1225, 1235. The release driver can be placed down either handle hole on the cage holder 1200. The driver is guided by the lumens or internal channels on the holder shaft and exits at the cage holder's distal tip 1205. In some examples, aperture 1266 includes a recess or counter bore 1267 with an axis offset from the axis of the aperture 1266 at the proximal end of the handle 1202. This may allow for a portion of a tool to be more easily inserted into the aperture 1266, or for tools to be simultaneously inserted into both apertures 1266 and 1268. For example, while the release driver is in place in lumen 1220, a bone screw delivery device 700 may be inserted through lumen 1230 to secure a first securement feature extending through the main body of the spinal implant, such as threaded apertures 1362, to an adjacent vertebra, for example using bone screw 708 engaging the threaded aperture 1362 and the adjacent bony surface, to fixedly secure the spinal implant within the spinal facet joint. The release driver and bone screw delivery device may be removed and a second bone screw delivery device 700 may be inserted through lumen 1220 to secure a second securement feature extending through the main body of the spinal implant, such as threaded apertures 1363, to an adjacent vertebra, for example using bone screw 708 engaging the threaded aperture 1363 and the adjacent bony surface, to fixedly secure the spinal implant within the spinal facet joint.

In some embodiments, the handle may further include an orientation mark or arrow to indicate the orientation for insertion and aid a new user with the order of deployment (lower lumen, then upper lumen) for ease of training and deployment consistency. However, the device can work in either orientation, and the two screws can be deployed in any order, depending on which lumen is used to retain the cage with the release driver.

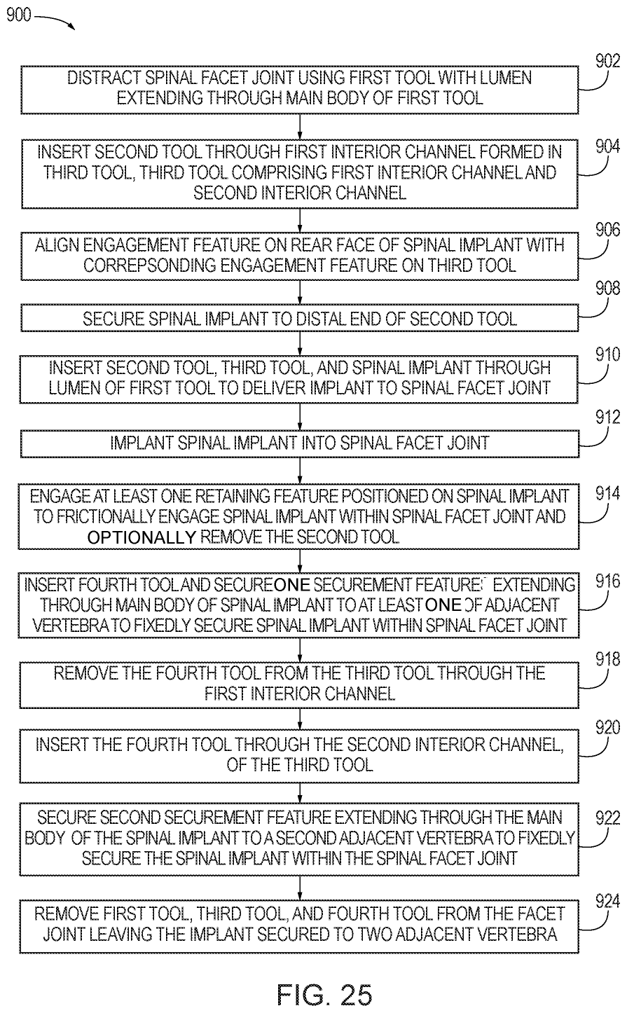

FIG. 25 illustrates a method 900 of fusing adjacent vertebrae in a spinal facet joint in accordance with one embodiment. The method may optionally include some or all of the steps and the steps may be performed in a different order. The method 900 may include step 902 to distract a spinal facet joint using a first tool, such as a guide tube 600, with a lumen extending through a main body of the first tool. In step 904, the method 900 may include inserting a second tool, such as a release driver 400, through a first interior channel formed in a third tool, such as a cage holder 200. The third tool may comprise the first interior channel 220 and a second interior channel 230. The method may include step 906 to align an engagement feature, such as features 340, 350 on a rear face 310 of a spinal implant 300 with a corresponding engagement feature, such as engagement features 240, 250 on the cage holder 200. In step 908, the method 900 may include securing the spinal implant 300 to a threaded distal end 404 of the release driver 400. In step 910, the method 900 may include inserting the second tool, such as the release driver 400, third tool, such as the cage holder 200, and spinal implant, such as implant 300, through the lumen of the first tool, such as the guide tube 600 to deliver the implant to a spinal facet joint. In step 912, the method 900 may include implanting the spinal implant into the spinal facet joint. In step 914, the method 900 may include engaging at least one retaining feature, such as engagement feature 330, positioned on the spinal implant to frictionally engage the spinal implant within the spinal facet joint and, optionally, removing the second tool, the release driver 400. In step 916, the method 900 may include inserting a fourth tool, such as a bone screw delivery device 700, and securing a first securement feature extending through the main body of the spinal implant, such as threaded apertures 362, to an adjacent vertebra, for example using bone screw 708 engaging the threaded aperture 362 and the adjacent bony surface, to fixedly secure the spinal implant within the spinal facet joint. If the second tool, the release driver 400 is not removed in step 914, then it may be removed at step 916 after the bone screw has secured the implant within the joint or during step 918.

In step 918, the method may include removing the fourth tool from the third tool through the first interior channel. In step 920, the method may include inserting the fourth tool through the second interior channel, such as channel 230, of the third tool, the cage holder. In step 922, the method may include securing the second securement feature, such as aperture 363, extending through the main body of the spinal implant to a second adjacent vertebra, for example using bone screw 708 engaging the threaded aperture 363 and the adjacent bony surface, to fixedly secure the spinal implant within the spinal facet joint. In step 924, the method may include removing the first tool such as the guide tube, third tool such as the cage holder, and fourth tool such as the bone screw delivery device from the facet joint and spinal surgical site, leaving the implant secured to two adjacent vertebra.

All relative and directional references (including: upper, lower, upward, downward, left, right, leftward, rightward, top, bottom, side, above, below, front, middle, back, vertical, horizontal, and so forth) are given by way of example to aid the reader's understanding of the particular embodiments described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other unless specifically set forth in the claims.

Those skilled in the art will appreciate that the presently disclosed embodiments teach by way of example and not by limitation. Therefore, the matter contained in the above description or shown in the accompanying drawings should be interpreted as illustrative and not in a limiting sense. Thus, it is intended that the scope of the present disclosure should not be limited by the particular embodiments described above.

What is claimed is:

1. A system for delivering a spinal implant into a spinal facet joint space via a posterior approach, the system comprising:
a spinal implant comprising first and second securement apertures configured to receive a fastener to fixedly secure the implant within the spinal facet joint;
a first tool defining two longitudinally extending lumens and comprising a proximal end and a distal end, the spinal implant received at the distal end;
a driver tool received in one of the two longitudinally extending lumens and configured to engage the spinal implant during delivery of the spinal implant to the spinal facet joint; and
a second tool defining a longitudinally extending shaft and the fastener,
wherein the second tool is received in one of the two lumens of the first tool to deliver the fastener to one of the securement apertures of the spinal implant.

2. The system of claim 1, further comprising a guide tube defining a longitudinally extending lumen and configured to receive at least the first tool to deliver the spinal implant to the spinal facet joint space.

3. The system of claim 1, wherein a third tool is received in the other of the two lumens of the first tool to deliver a second fastener to the other of the two securement apertures of the spinal implant.

4. The system of claim 3 wherein the fastener is a bone screw, and the second tool and/or the third tool includes a breakable junction between the longitudinally extending shaft and the bone screw such that the bone screw detaches from the shaft after deployment in one of the securement apertures of the spinal implant.

5. The system of claim 1, the implant further comprising:
a main body comprising opposing top and bottom surfaces, opposing front and rear surfaces, opposing side surfaces, and having a longitudinal axis extending through the main body;
the first securement aperture extending through at least a portion of the rear and top surface and the second securement aperture extending through at least a portion of the rear and bottom surface; and
the first and second securement apertures extending through the main body at offset angles relative to the longitudinal axis of the main body.

6. The system of claim 5, the implant further comprising:
at least one engagement feature defined at least partially in the rear surface and at least one of the top or bottom surface of the main body, the engagement feature configured to receive a corresponding engagement feature on the first tool.

7. The system of claim 6, wherein the at least one engagement feature defined on the main body is a keyway feature and the corresponding engagement feature on the first tool is a corresponding key.

8. The system of claim 6, wherein the at least one engagement feature is a symmetrical groove, an asymmetrical groove or indentation that is complementary to at least one engagement feature on an implant delivery device.

9. The system of claim 6, wherein the at least one engagement feature is a protrusion in the rear surface that is complementary to at least one engagement feature on an implant delivery device.

10. The system of claim 5, the implant further comprising:
at least one retaining feature associated with at least one of the top or bottom surfaces of the main body to frictionally engage the implant within the spinal facet joint, wherein the at least one retaining feature includes a trailing face spaced apart from the rear surface of the implant.

11. A spinal implant for implantation within a spinal facet joint, the implant comprising:
a main body including:
opposing top and bottom surfaces;
opposing front and rear surfaces;
opposing side surfaces; and
a longitudinal axis extending through the main body;
at least one retaining feature associated with at least one of the top or bottom surfaces of the main body to frictionally engage the implant within the spinal facet joint;
a first securement aperture extending through at least a portion of the top surface and a second securement aperture extending through at least a portion of the bottom surface, the first and second securement apertures extending through the main body at offset angles relative to the longitudinal axis of the main body; and
at least one engagement feature defined at least partially in the rear surface and at least one of the top or bottom surface of the main body, the engagement feature configured to receive an implant delivery device.

12. The spinal implant of claim 11, wherein the two securement apertures are operable to receive fasteners, preferably a bone screw.

13. The spinal implant of claim 11, the implant further comprising at least one window, and preferably two windows, defined in the top and/or bottom surface of the main body.

14. The spinal implant of claim 13, the implant further comprising one window defined in each side surface of the main body.

15. The spinal implant of claim 11, wherein the at least one engagement feature is a keyway.

16. The spinal implant of claim 11, wherein the at least one engagement feature is a symmetrical groove, an asymmetrical groove or indentation that is complementary to features on an implant delivery device.

17. A system for delivering a spinal implant into a spinal facet joint space via a posterior approach, the system comprising:
a spinal implant according to claim 11;
a first tool defining two longitudinally extending lumens and comprising a proximal end and a distal end, the spinal implant received at the distal end;
a driver tool received in one of the two longitudinally extending lumens and configured to engage the spinal implant during delivery of the spinal implant to the spinal facet joint space; and
a second tool defining a longitudinally extending shaft and a fastener,
wherein the second tool is received in one of the two lumens of the first tool to deliver the fastener to one of the securement apertures of the spinal implant.

18. The system of claim 17, wherein a third tool is received in the other of the two lumens of the first tool to deliver a second fastener to the other of the two securement apertures of the spinal implant.

19. The system of claim 17, wherein the fastener is a bone screw, and the second tool and/or the third tool includes a breakable junction between the longitudinally extending shaft and the bone screw such that the bone screw detaches from the shaft after deployment in one of the securement apertures of the spinal implant.

20. A method of fusing adjacent vertebra defining a spinal facet joint, the method comprising:
distracting a spinal facet joint using a first tool with a lumen extending through a main body of the first tool;
inserting a second tool through a first interior channel formed in a third tool, the third tool comprising the first interior channel and a second interior channel;
aligning an engagement feature on a rear face of a spinal implant with a corresponding engagement feature on the third tool;
securing the spinal implant to a distal end of the second tool;
inserting the second tool, third tool, and spinal implant through the lumen of the first tool to deliver the implant to a spinal facet joint;
implanting a spinal implant into the spinal facet joint;
engaging at least one retaining feature positioned on the spinal implant to frictionally engage the spinal implant within the spinal facet joint and removing the second tool; and
inserting a fourth tool and securing two securement features extending through the main body of the spinal implant to at least two of the adjacent vertebra to fixedly secure the spinal implant within the spinal facet joint.

21-25. (canceled)
